(19) United States
(12) Patent Application Publication
(10) Pub. No.: US 2016/0038627 A1
(43) Pub. Date: Feb. 11, 2016

(54) METHOD OF CAUSING DELAYED HEMOSTASIS

(71) Applicant: St. Teresa Medical Inc., Woodbury, MN (US)

(72) Inventors: Curtis Olson, St. Paul, MN (US); Philip Messina, Woodbury, MN (US)

(21) Appl. No.: 14/819,051

(22) Filed: Aug. 5, 2015

Related U.S. Application Data
(60) Provisional application No. 62/034,015, filed on Aug. 6, 2014.

(51) Int. Cl. A61L 15/44; A61L 15/28; A61L 15/56

(52) U.S. Cl. CPC A61L 15/44; A61L 15/56; A61L 15/28; A61L 2300/418; A61L 2300/442

(57) ABSTRACT

A method of causing delayed hemostasis. A hemostatic product is formed by applying a hemostatic agent to a dextran support. The hemostatic agent is provided with at least one of a reduced concentration and a reduce availability. The hemostatic product is applied to a wound from which blood is being discharged. At least one foreign object or pathogen is proximate the wound. Hemostasis is progressively caused so that blood continues to be discharged from the wound to cause the at least one foreign object or pathogen to move away from the wound.

### FIELD OF THE INVENTION

This application claims priority to U.S. Provisional Application No. 62/034,015, which was filed on Aug. 6, 2014, the contents of which are incorporated herein by reference.

### FIELD OF THE INVENTION

The invention relates generally to products having hemostatic characteristics. More particularly, the invention relates to stabilizers for use in hemostatic products.

### BACKGROUND OF THE INVENTION

The body's natural response to stem bleeding from a wound is to initiate blood clotting via a complex process known as the coagulation cascade. The cascade involves two pathways that ultimately lead to the production of the enzyme thrombin, which catalyzes the conversion of fibrinogen to fibrin.

Fibrin is then cross-linked to form a clot, resulting in hemostasis. For wounds that are not severe, and in individuals that have no countervening conditions, the body is usually able to carry out this process efficiently in a manner that prevents excessive loss of blood from the wound. However, in the case of severe wounds, or in individuals in whom the clotting mechanism is compromised, this may not be the case.

For such individuals, it is possible to administer components of the coagulation cascade, especially thrombin and fibrinogen, directly to the wound to bring about hemostasis. Bandaging of bleeding wounds is also a usual practice, in part to isolate and protect the wounded area, and also to provide a means to exert pressure on the wound, which can also assist in controlling bleeding.

While these methods may be carried out satisfactorily in cases of mild trauma or under conditions of “controlled” wounding (e.g. surgery), many situations in which such treatments are most needed are also those in which it is the most difficult to provide them. Examples of such wounds include, for example, those inflicted during combat or unanticipated wounds that occur as the result of accidents. In such circumstances, survival of the wounded individual may depend on stopping blood loss from the wound and achieving hemostasis during the first few minutes after injury. Unfortunately, given the circumstances of such injuries, appropriate medical intervention may not be immediately available.

In particular, the treatment of penetrating wounds such as bullet wounds or some wounds from shrapnel is problematic. This is due to the difficulty in placing a hemostatic product and/or therapeutic agents at the actual site of injury, which includes an area that is well below the body surface and difficult or impossible to access using conventional techniques.

Jiang et al. in Biomacromolecules, v. 5, p. 326-333 (2004) teaches electrospun dextran fibers. Agents associated with the fibers (e.g. BSA, lysozyme) are directly electrospun into the fibers. The fibers may also include other polymers electrospun with the dextran.

Jiang et al. in Journal of Biomedical Materials Research Part B: Applied Biomaterials, p. 50-57 (2006) discloses electrospun fibers that are a composite of poly(c-caprolactone) as a shell and dextran as a core. These fibers provide the slow release of agents (bovine serum albumin, BSA) that are also electrospun into the fibers.

Smith et al., U.S. Pat. No. 6,753,454, discloses electrospun fibers comprising a substantially homogeneous mixture of a hydrophilic polymer and a polymer that is at least weakly hydrophobic, which may be used to form a bandage. The bandage may comprise active agents (e.g. dextran). However, the disclosed fibers are not readily soluble in liquid.

MacPhee et al., U.S. Pat. No. 6,762,336, teaches a hemostatic multilayer bandage that comprises a thrombin layer between two fibrinogen layers. The bandage may contain other resorbable materials such as glycolic acid or lactic acid based polymers or copolymers. Neither electrospun fibers nor dextran fibers are taught as components of the bandage.

Smith et al., U.S. Pat. No. 6,821,479, teaches a method of preserving a biological material in a dry protective matrix, the matrix comprising fibers such as electrospun fibers. One component of the fibers may be dextran, but homogeneous dextran fibers are not described.

Cochrum et al., U.S. Pat. No. 7,101,862, teaches hemostatic compositions and methods for controlling bleeding. The compositions comprise a cellulose-containing article (e.g. gauze) to which a polysaccharide is covalently or ionically crosslinked. The crosslinked polysaccharide may be dextran. However, the compositions are not electrospun and exogenous clotting agents are not included in the compositions.

Wnek et al., U.S. Patent Publication No. 2004/0018226, discloses fibers produced by an electroprocessing technique such as electrospinning The fibers comprise enclosures within the fibers for containing substances that are not miscible with the fibers. Dextran is not taught as a fiber component.

Fisher et al., U.S. Patent Publication No. 2007/0160653, teaches a hemostatic textile comprising hemostatic factors (e.g. thrombin, fibrinogen) but the fibers are formed from electrospun glass plus a secondary fiber (e.g. silk, ceramic, bamboo, jute, rayon, etc.).

Carpenter et al., U.S. Patent Publication No. 2008/0020015, teaches wound dressing comprised of various biodegradable polymers and hydrogels having allogenic or autologous precursor cells (e.g. stem cells) dispersed within the polymers. The polymers may be prepared by electrospinning, and one polymer component may be dextran. However, the polymers cannot be immediately soluble upon contact with liquid, as they must provide a scaffolding for delivery of the cells over time, even though the polymers eventually biodegrade in situ.

Li et al., U.S. Patent Publication No. 2008/0265469, describes electrospun nanofibers that may include dextran. However, the nanofibers are not described as readily soluble in liquids.

Eskridge et al., U.S. Patent Publication No. 2009/0053288, teaches a woven hemostatic fabric comprised of about 65% fiberglass yarn and about 35% bamboo yarn. The fiberglass component may be electrospun, and hemostatic factors such as thrombin may be associated with the fabric, e.g. by soaking the material in a solution of thrombin. This document indicates that dextran may be added as a hygroscopic agent.

There is an ongoing need to provide improved methods and means to initiate blood clotting in wounds to stop or at least slow blood loss. In particular, there is an ongoing need to improve the capability to readily promote hemostasis in severe wounds in a facile manner, especially under circumstances where immediate treatment by medical personnel is limited or unavailable.

Bowlin et al., U.S. Patent Publication No. 2011/0150973, discloses a method of delivering one or more agents of interest to a location of interest. The method includes applying or delivering to a location of interest a hemostatic product. The hemostatic product includes electrospun dextran fibers that dissolve upon contact with liquid. The hemostatic product also includes one or more agents of interest associated with said electrospun dextran fibers. Applying or delivering results in dissolution of the electrospun dextran fibers in liquid at the location of interest to thereby release the one or more agents of interest into the liquid.

### SUMMARY OF THE INVENTION

An embodiment of the invention is directed to a method of causing delayed hemostasis. A hemostatic product is formed by applying a hemostatic agent to a dextran support. The hemostatic agent is provided with at least one of a reduced concentration and a reduce availability. The hemostatic product is applied to a wound from which blood is being discharged. At least one foreign object or pathogen is proximate the wound. Hemostasis is progressively caused so that blood continues to be discharged from the wound to cause the at least one foreign object or pathogen to move away from the wound.

Another embodiment is directed to a method of performing a surgical procedure. A hemostatic product is formed that includes a dextran support and a hemostatic agent. The hemostatic agent is associated with the electrospun dextran base. A bone is cut to change a shape of the bone. Cutting the bone causes blood to be discharged from the bone. The hemostatic product is applied to the bone proximate to where blood is being discharged from the bone to at least partially stop the blood flow. The shape of the bone is evaluated.

Another embodiment is directed to a hemostatic product that includes a dextran support, a hemostatic agent and a diagnostic agent. The hemostatic agent is associated with the dextran support. The diagnostic agent is associated with the dextran support.

### DETAILED DESCRIPTION OF THE PREFERRED EMBODIMENTS

An embodiment of the invention is directed to a system for providing hemostasis in a person or animal. When the hemostatic product is applied to the injury site, the materials used to fabricate the hemostatic product dissolve to thereby release the materials to the injury site and provide the hemostatic effect.

In some embodiments of the invention, only electrospun dextran fibers are utilized and thus after clot formation, there is no need to disturb the clot to remove hemostatic product components, since none remain at the site. The hemostatic product thereby does not leave any residual foreign bodies that elicit foreign body reactions or act as a nidus for infection. Furthermore, the hemostatic product does not contain any xenoproteins, which have the potential of eliciting immune reactions in persons on which the hemostatic product is used.

In other embodiments, as described below, the hemostatic product may include other materials such as support or backing material, which, after initial rapid application of the hemostatic product, may later be removed for further treatment of the wound by conventional methods.

The system generally includes a hemostatic product having a base to which at least one hemostatic agent is associated. In certain embodiments, the base is fabricated from electrospun dextran and the hemostatic agent is thrombin and/or fibrinogen.

Electrospinning is a non-mechanical processing strategy and can be scaled to accommodate the large volumes necessary to meet the needs of commercial processing. Additional details on the electrospinning process are provided in U.S. application Ser. No. 12/937,322, the contents of which are incorporated herein by reference.

In certain embodiments, the base used in the hemostatic products is formed of substantially homogeneous spun dextran. The amount of dextran used in each hemostatic product can vary depending on the size of hemostatic product that is being manufactured, with typical hemostatic product formulations using from about 5-10 grams of dextran (usually 100,000-200,000 Mr) per hemostatic product.

Of more consequence is the concentration of dextran in the solution from which the fibers are electrospun. Generally, a solution of dextran for electrospinning will be of a concentration in the range of between about 0.1 and about 10 grams per milliliters of solvent. In other embodiments, the dextran concentration is between about 0.5 and about 5 grams per milliliter, and usually such a solution is at a concentration of about 1 gram per milliliter, which is about 0.15 milligrams. A preferred range would be from about 0.9 to about 1.1 grams of dextran per milliliter of solution that is to be spun.

Those of skill in the art will recognize that a variety of liquid solvents exist in which it is possible to dissolve dextran. However, superior results for electrospinning dextran are generally achieved when the solvent is water, especially deionized or distilled or deionized, distilled (ddH₂O) or other forms of relatively pure water. In addition, there are no negative interactions during use of the hemostatic product associated with water remaining in the hemostatic product and there is far less environmental impact associated with the use of water as compared to many other solvents.

The area (length and width) of the hemostatic product of the invention can vary widely and can be adjusted by adjusting spinning parameters. In addition, the mats of dextran fibers can be cut to a desired size after spinning Generally, the hemostatic product will be from about 0.5 centimeters or less to about 30 centimeters or more in length and/or width, but larger or smaller sizes are also contemplated.

The thrombin and/or fibrinogen that are associated with the hemostatic product are in forms that are biologically active when they come into contact with blood. Hence, upon dissolution, the thrombin acts on the fibrinogen, converting it to fibrin, which then forms a clot within the wound to thereby staunch the flow of blood.

In certain embodiments, the thrombin and fibrinogen may be derived from human sources. In other embodiments, the thrombin and fibrinogen are salmon thrombin and fibrinogen. Advantages of using salmon as a source of these materials include but are not limited to the lack of concern about transmission of etiologic agents (e.g. viruses) that may occur when human and other mammalian sources of thrombin or fibrinogen (e.g. bovine) are used.

The quantity of fibrinogen added to the hemostatic product is generally in the range of from about 10 milligrams to about 3 grams. In certain embodiments, the amount of fibrinogen in each of the hemostatic products is between about 20 milligrams to about 1 gram.

The quantity of thrombin added to each of the hemostatic products is generally between about 10 and 10,000 NIH Units. In certain embodiments, the amount of thrombin in each of the hemostatic products is between about 20 and 6,000 NIH Units.

In other embodiments, the amount of the hemostatic agent in the hemostatic product is reduced so that upon release of the hemostatic agent, hemostasis is not promptly obtained. A benefit of such configuration is that the continued bleeding from the wound provides sufficient time for the flowing blood to wash foreign objects from the wound.

While the duration of the controlled bleeding should be sufficiently long to cause a large portion of the foreign objects in the wound to be washed out, the duration of the controlled bleeding should not be too long to minimize potential negative effects caused by the patient loosing too much blood.

Another way to control the rate at which the hemostatic agent is released from the hemostatic product is to divide the hemostatic agent between multiple layers of the electrospun dextran support. Yet another technique to control the rate at which the hemostatic agent is released from the hemostatic product is to increase the density of the hemostatic product such as by compressing the hemostatic product.

In some embodiments, the therapeutic agents may themselves be electrospun. For example, the therapeutic agents are dissolved in and spun from a solution. In some embodiments, the therapeutic agents may be electrospun into fibers. In other embodiments, the active agents may be electrospun into other forms such as droplets, beads, etc.

In some applications, active agents such as thrombin may be electrosprayed with sucrose to form sugar droplets, which tends to stabilize thrombin and can also “trap” other substances of interest for delivery to the hemostatic product.

For thrombin and fibrinogen, in most embodiments, these (or other) active agents are in a finely dispersed dry, particulate or granular form e.g. as a fine powder or dust, as electrospinning may tend to decrease their activity. In other words, the active agents are not electrospun either by themselves.

Usually the agents are bioactive agents that have a beneficial or therapeutic effect at the wound site. In one embodiment, the site is a bleeding wound at which it is desired to form a blood clot to stop or slow the bleeding. In this embodiment, the therapeutic substances of interest may include, for example, thrombin and fibrinogen, although other agents active in promoting hemostasis, including but not limited to capscian, may also be included.

In addition, electrospun or non-electrospun collagen, agents that absorb water, various dry salts that would tend to absorb fluids when placed in contact with e.g. blood; engineered thrombin or thrombin mimics; engineered fibrinogen; agents that cause vasospasm (e.g. ADP, 5-hydroxytryptamine, 5-HT and thromboxane, (TXA-2) to help contract and seal a bleeding vessel, etc. may also be included.

Other components of the clotting cascade may be added to the hemostatic product, for example: tissue factors that are normally only expressed on the surface of damaged cells and that start the normal clotting cascade; serotonin which enhances platelet clumping and promotes vessel constriction; and other agents that are used to replace missing components of the clotting cascade in hemophilia, for example, factor 7 (which activates the so called external extrinsic coagulation cascade) and crude extracts of platelets.

Active agents that function to promote late stages of wound healing may also be included to, for example, facilitate cell migration and remodeling. The incorporation of collagen is an example of such an active agent.

One or more of any of these active agents may be used in the practice of the present invention. The therapeutic agents must be amenable to drying and are associated with the other components of the hemostatic product in the dry state, since liquid may negatively affect at least one of the components used in the hemostatic product. For example, the active agents may be desiccated or lyophilized, or water may be removed by other means.

Association of substances of interest with the electrospun dextran base may be accomplished by any of many suitable techniques that are known to those of skill in the art, and will depend in part on the precise form of the substance and the means at hand. For example, for powdered, particulate thrombin and fibrinogen, association may be carried out by sprinkling, shaking, blowing, etc. the agents onto a layer of the excipient or carrier.

In certain embodiments, the electrospun dextran base is placed on a vacuum table, which not only retains the electrospun dextran base in a substantially stationary position during the fabrication process but also causes the hemostatic agents to be drawn into the electrospun dextran base. This process thereby reduces the potential of the hemostatic agent becoming disassociated from the electrospun dextran base while stored in a package as well as when removed from the package prior to applying to the wound.

Depending on the density of the fiber mat, the substances of interest may become relatively evenly dispersed throughout the woven mat of fibers or may be largely confined to the topmost section of the fiber mat. If no backing is present, the latter embodiment is preferable to prevent the particulate substance of interest from falling through and out of the mat.

The association of substances of interest with the electrospun dextran base may be carried out according to many different arrangements. For example, a first layer of electrospun dextran may be formed, and one or more of the substances may be associated with the first layer. Then another second layer of electrospun dextran may be formed on top of the substance(s) of interest, and the same or other substances of interest may be associated with the second layer, and so on.

A final or outermost layer of electrospun dextran may be added to prevent the dislodgement of substances of interest from the layer(s) below. The number of layers of excipient that are used in the hemostatic product of the invention may vary widely, from as few as 1-2 to as many as several dozen, or even several hundred, depending on the desired characteristics of the hemostatic product.

Typically, a hemostatic product will contain 1-2 layers. In other embodiments the hemostatic product may include between 2-20 layers. The very slight amount of moisture that is present in a prepared hemostatic product may help to trap and retain the thrombin and fibrinogen on the surface of the hemostatic product.

The height or thickness of the hemostatic product can vary considerably depending on the intended use of the hemostatic product. In certain embodiments, the hemostatic product has a thickness of between about 1 millimeter and about 5 centimeters.

The thickness of the hemostatic product (which is related to the volume) may impact the rate of dissolution of the dextran upon contact with liquid. For example, a thin hemostatic product (e.g. about 2 millimeters) will dissolve more rapidly than a hemostatic product that is thicker, providing the loft of the fibers is comparable.

In most embodiments, dissolution of the dextran fibers is extremely rapid, e.g. about 5 minutes or less after exposure to liquid, or about 4 minutes or less, or about 3 minutes or less, or about 2 minutes or less, or about 1 minute or less. In certain embodiments, the hemostatic product substantially dissolves in between about 1 second and about 20 seconds.

This rapid dissolution may be referred to herein as “instantaneous” or “immediate” dissolution. Compression of an electrospun dextran mat may be used to modulate the rate of dissolution, with greater levels of compression inversely impacting the rate, i.e. generally, the greater the degree of compression, the slower the rate of dissolution.

The rapid rate of dissolution is advantageous, particularly when delivering biologically active agents (e.g. hemostatic agents) to a site of action such as a wound. Rapid dissolution of the carrier dextran fibers provides extremely rapid delivery of the hemostatic agents to the wound upon deployment of the hemostatic product.

Generally, the amount of water that is present in the substances when they are associated with the electrospun dextran fibers is less than about 5%, and preferably less than about 2%. These substances retain full or partial activity when rehydrated, e.g. in blood. Generally, therapeutic substances associated with the hemostatic products of the invention retain, upon contact with liquid, at least about 25%, or about 50%, or even about 75% to 100% of their activity before drying or desiccation, as compared to standard preparations of the substance using standard assays that are known to those of skill in the art.

If thrombin is included in the hemostatic product, it may be desirable to reduce the moisture content of the hemostatic product (e.g. a bandage or gauze) to less than about 5% to preserve thrombin activity during sterilization.

This moisture content reduction can be achieved by drying the fabricated hemostatic product, e.g., under a vacuum, or by using a fabrication method that reduces moisture content from the beginning

The hemostatic product may include one or more stabilizers such as is described in U.S. application Ser. No. 13/622,690, which is assigned to the assignee of the present application and the contents of which are incorporated herein by reference. The stabilizers may enhance the ability of the hemostatic product to dissolve when the hemostatic products are applied to the injury site.

Prior to use of the hemostatic product, it may be desirable for the hemostatic product to be carried by a person on whom the hemostatic product could potentially be used and/or by a person who could potentially use the hemostatic product. In other embodiments, the hemostatic product resists degradation at temperatures of more than 140° F. to less than 0° F.

In certain embodiments, the hemostatic product should resist degradation when exposed to the elevated temperature such as up to about 150° F. for more than about 3 hours. In other embodiments, the hemostatic product should resist degradation when exposed to the elevated temperature for up to about 24 hours.

A threshold for the hemostatic product to be viewed as not experiencing degradation is that the hemostatic product does not exhibit noticeable visible physical changes when viewing the hemostatic product without magnification. The hemostatic product should also not experience noticeable physical changes when the hemostatic product is examined with magnification such as with a magnifying glass or a microscope.

The preceding characteristics should be displayed by the hemostatic product regardless of whether the hemostatic product is retained in the packaging materials while exposed to the elevated temperature conditions.

The stabilizer also enhances the usable shelf life of the hemostatic product. In certain embodiments, the stabilizer provides the hemostatic product with a shelf life of at least about 2 years. In other embodiments, the hemostatic product exhibits a shelf life of at least 3 years. As used herein, the term usable shelf life means that the hemostatic product does not exhibit noticeable degradation when viewed without magnification or with magnification such as a magnifying glass or microscope.

To minimize the potential of degradation of the hemostatic product, the hemostatic product should be protected from exposure to moisture because when the components used in the hemostatic product are exposed to moisture, the components degrade such as by dissolving.

In some embodiments of the invention, the hemostatic products also include one or more support structures or support materials incorporated therein. For example, a backing may be incorporated into the hemostatic product.

The support material may be formed from various electrospun materials such as polyglycolic acid (PGA), polylactic acid (PLA), and their copolymers (PLGAs); charged nylon, etc. In one embodiment, the support material is compressed electrospun dextran fibers. By “compressed electrospun dextran fibers,” it is meant that electrospun dextran fibers are compressed together under pressure.

The support material may or may not be soluble in liquid, or may be slowly soluble in liquid, and may or may not be permeable to liquid. Slowly soluble materials include those from which absorbable or dissolving (biodegradable) stitches or sutures are formed, included PGA, polylactic and caprolactone polymers.

In certain embodiments, the support material may dissolve relatively quickly such as less than about 1 hour. In other embodiments, the support material may dissolve within from about 10 days to 8 weeks. In either case, the support material provides the advantage of not having to remove the hemostatic product and risk disrupting the clot.

However, in any case, the support material should not interfere with the immediate dissolution of the hemostatic product and delivery of the active agents associated therewith into the liquid that dissolves the hemostatic product. All such arrangements, shapes, and embodiments of carrier layers and support materials as described herein are intended to be encompassed by the invention.

The hemostatic product may be sterilized prior to use, generally by using electromagnetic radiation, for example, X-rays, gamma rays, ultraviolet light, etc. Typically, the hemostatic products are sterilized using X-rays in a dose of about 5 kilograys (kGray). Any method that does not destroy the carrier or the activity of substances associated with the fibers may be used to sterilize the hemostatic products of the invention.

The hemostatic product may also include diagnostic agents that can be used by the treating medical professional to diagnose the nature of the injury. In certain embodiments, the diagnostic agent may change colors to indicate the presence of particular chemicals in the blood or to indicate particular characteristics of the blood. For example, if the patient is currently taking medications that cause thinning of the patient's blood. The diagnostic agents could also change colors to indicate the oxygen and/or glucose level of the blood.

In other embodiments, the products of the invention need not comprise agents that promote clotting at all. Those of skill in the art will recognize that the products of the invention are highly suitable for delivering many substances of interest to a desired liquid environment or location. For example, the products may be designed for delivery of therapeutic or beneficial substances to any moist environment of the body, where there is sufficient liquid to dissolve the electrospun dextran fibers and release the active substance, and where dissolved dextran is not problematic.

Such substances may include, for example, enzymes or their precursors (e.g. pro-enzymes or zymogens) and their substrates, substances that activate a protein or enzyme (e.g. proteases, cofactors, etc.), and the like.

For example, hemostatic products comprised of only thrombin might be used for small injuries or in combination with other interventions. In addition, other therapeutically beneficial substances may also be associated with the hemostatic product, including but not limited to: antibiotics, antiviral agents, anti-helminthic agents, anti-fungal agents, medicaments that alleviate pain, growth factors, bone morphogenic protein, vasoactive materials (e.g. substances that cause vasospasms), steroids to reduce inflammation, chemotherapy agents, contraceptives, etc.

Examples include but are not limited to oral, nasal, tracheal, anal, lung, and vaginal delivery of substances such as anti-microbial agents, analgesic agents, nutritional agents, etc. Oral applications include the delivery of substances useful for dental treatments, e.g. antibiotics, pain medications, whitening agents, etc.

In some embodiments, no bodily fluid is present (or if insufficient body fluid is present) and the applied hemostatic product can be “activated” by wetting, e.g. by spraying, or by otherwise applying a source of moisture (e.g. by exposing the hemostatic product to a moist material such as a sponge), or dropping hemostatic products into a liquid (e.g. a body of water), to cause release of the agents of interest associated with the dextran fibers.

In certain embodiments, the hemostatic product is used in conjunction with spinal or joint replacement surgical procedures. When performing certain spinal operations such as spinal vertebra fusion, it is necessary to cut into or otherwise disturb the surface of the bone. This process results in cancellous or cortical bone bleeding.

The cancellous bone is highly vascularized and, as such, can result in significant bleeding when the cancellous bone is cut or the surface thereof is otherwise disturbed. This cancellous or cortical bone bleeding impedes the ability to accurately visualize the bone surface. Accurately visualizing the bone surface enhances the ability to accurately position an implant with respect to the bone as well as to accurately place fixation devices with respect to the bone.

After the bone is cut, the hemostatic product is applied to the surface thereof. Similar to the other uses of the hemostatic product described herein, the contact of liquid such as from blood with the hemostatic product causes rapid dissolution of the electrospun dextran, which thereby releases the hemostatic agent to cause hemostasis. Once hemostasis is achieved, it is possible to accurately position bones and/or fastening devices with respect to the bones.

Yet another type of surgery that produces significant amount of bleeding is thoracic surgery. For example, surgery on organs such as heart, lungs and esophagus can result in bleeding that presents a challenge to successfully completing the surgery.

Another orthopedic application for the hemostatic product is in conjunction with bone grafts. In such surgeries, cutting or otherwise disturbing the surface of the bone where it is desired to place the bone graft can cause significant bleeding, which impacts the ability to accurately place the bone graft and degrades the ability of the bone graft to become associated with the native bone.

In many applications, the bone graft is provided in a powder and significant blood flow can cause the bone graft powder to flow from the region where it is desired to be used. Applying the hemostatic product to the cut bone can reduce or eliminate bleeding to thereby facilitate not only accurate placement of the bone graft but also minimize the potential of the bone graft prematurely moving from the desired location.

Another area in which it can be challenging to achieve hemostasis is when a catheter is introduced into an artery or vein such as the femoral artery. In certain embodiments, the hemostatic product is applied after the catheter has been introduced to stop bleeding from around the catheter. In another embodiment, the hemostatic product is formed into a sheet, which is wrapped around the catheter proximate to where the catheter is intended to pass into the artery or vein.

Another area in which bleeding presents a challenge to successfully treating a patient is when performing dental procedures. Examples of types of bleeding that are commonly experienced when performing dental procedures include maxillary, mandibular, gum, palate and tongue bleeding. For example, moisture present in a person's mouth can present challenges to causing hemostasis.

The hemostatic product can be formed into strips having a relatively small size that facilitate placement into portions of the mouth that are rather confined. In situations where one of the hemostatic strips is not sufficient to cause a desired degree of hemostasis, multiple hemostatic strips can be used.

The electrospun dextran base can be formed into the shape of a generally cylindrical plug to facilitate placement of the hemostatic product into openings in the patient's mouth such as when a tooth is removed. The hemostatic agents can either be applied to the surface of the plug or can be applied to the electrospun dextran before the electrospun dextran is formed into the plug. While the later approach may increase the time necessary to achieve hemostasis, this approach may promote forming a stronger clog that is less likely to be disturbed and, therefore, is less likely to result in rebleeding.

Because of the combined liquid from blood and moisture in the mouth, there is typically sufficient liquid to cause the electrospun dextran to dissolve. In situations where there is not sufficient liquid to cause substantially all of the electrospun dextran to dissolve, it is possible for additional moisture to be introduced proximate to where the hemostatic product is applied to the wound. An example of one suitable technique is by dispersing saline from a syringe. An advantage of this technique is that it enables the liquid to be accurately dispensed toward the hemostatic product.

Certain tumors are associated or otherwise attached to bone. Efforts to extract the tumors may result in significant bleeding. It is desired to remove substantially all of the tumor while minimizing damage to the bone plays a significant role in successfully treating the patient especially in view of the fact that the prior growth of the tumor may have weakened the bone.

In such situations, the surgeon may endeavor to cut a significant portion of the tumor and then utilize the hemostatic product to stop or substantially slow the bleeding to thereby enable the surgeon to evaluate whether additional portions of the tumor need to be removed while minimizing inadvertent cutting of the bone.

Yet another type of bleeding that may present challenges to quickly causing hemostasis is in conjunction with biopsies. Examples of such biopsies include liver, bone and soft tissue. Currently, a cotton tip swab that is coated with a clotting agent is applied to the area where the biopsy was obtained. A drawback of this approach is that removing the swab can cause the clot to be disturbed, which results in rebleeding.

In certain procedures electrodes are applied to a patient and a portion of the electrode pierces the patient's skin. The hemostatic product is used in conjunction with such electrodes to stop bleeding that occurs either when the electrode is applied to the patient's skin or after the electrode is removed from the patient's skin.

In the former situation, the hemostatic product is applied to a portion of the electrode that is adjacent to the needle or other device that pierces the patient's skin such as are used in conjunction with spine shock therapy. The hemostatic product may be formed in the shape of a relatively thin sheet. Similar to the other embodiments described herein, the hemostatic may include an electrospun dextran base to which the hemostatic agents are applied.

In the later situation, the hemostatic product may be separate from the electrode and then applied to the wound caused by the needle or other device that pierces the patient's skin immediately after the electrode is detached from the patient's skin. Alternatively or additionally, the hemostatic product may be placed on an inner surface of the electrode but not attached to the electrode so that the hemostatic product remains on the patient's skin when the electrode is removed from the patient's skin.

The hemostatic product should remain sufficiently close to the wound so that the patient or caregiver does not need to move the hemostatic product to achieve hemostasis. In certain embodiments, an adhesive may be applied to an inner surface of the hemostatic product to enhance the ability of the hemostatic product to remain in place over the wound after the electrode is detached from the patient's skin.

It is also possible for an adhesive to be utilized between the hemostatic product and the electrode to maintain the hemostatic product in a desired position with respect to the electrode prior to when the electrode is applied to the patient's skin. In such situations, the strength of the adhesive on the side of the hemostatic product that faces the electrode is less than the strength of the adhesive on the side of the hemostatic product that faces the patient's skin so that the hemostatic product remains on the wound when the electrode is detached from the patient's skin.

The hemostatic product utilized in this embodiment may include an electrospun dextran base that is relatively thin. The hemostatic agent may be applied to an inner surface of the electrospun dextran base to reduce the potential of the hemostatic agent becoming dislodged from the electrospun dextran base. In an alternative embodiment, the hemostatic agent may be placed between two layers of the electrospun dextran base.

To reduce the potential of the hemostatic product moving with respect to the catheter, a relatively small amount of water may be applied to the surface of the catheter before the hemostatic product is applied to the catheter.

In addition to being used to produce hemostasis in humans, the concepts of the invention may be adapted for use in conjunction with other animals. Examples of such animals on which the invention can be used include dogs and cats.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A method of causing delayed hemostasis comprising:
forming a hemostatic product comprising applying a hemostatic agent to a dextran support, wherein the hemostatic agent is provided at least one of a reduced concentration and a reduce availability;
applying the hemostatic product to a wound from which blood is being discharged, wherein at least one foreign object or pathogen is proximate the wound; and
progressively causing hemostasis so that blood continues to be discharged from the wound to cause the at least one foreign object or pathogen to move away from the wound.

2. The method of claim 1, wherein moving the at least one foreign object or pathogen away from the wound reduces negative effects caused by the presence of the at least one foreign object or pathogen in the wound.

3. The method of claim 1, wherein the progressively causing hemostasis is caused by reducing a concentration of the hemostatic agent that is used to promptly cause hemostasis upon application of the hemostatic product to the wound.

4. The method of claim 1, wherein the progressively causing hemostasis is caused by compressing the hemostatic product.

5. The method of claim 1, wherein the dextran support comprises electrospun dextran and wherein the hemostatic agent comprises at least one of thrombin and fibrinogen.

6. A method of performing a surgical procedure comprising:
forming a hemostatic product that comprises a dextran support and a hemostatic agent that is associated with the electrospun dextran base;
cutting a bone to change a shape of the bone, wherein cutting the bone causes blood to be discharged from the bone;
applying the hemostatic product to the bone proximate to where blood is being discharged from the bone to at least partially stop the blood flow; and
evaluating the shape of the bone.

7. The method of claim 6, wherein the dextran support comprises electrospun dextran and wherein the hemostatic agent comprises at least one of thrombin and fibrinogen.

8. The method of claim 6, and further comprising attaching the dextran support to a support material that is selected from the group consisting of gauze, electrospun dextran, polyglycolytic acid polymers, polylactic acid polymers, caprolactone polymers and charged nylon.

9. A hemostatic product comprising:
a dextran support;
a hemostatic agent associated with the dextran support; and
a diagnostic agent associated with the dextran support.

10. The hemostatic product of claim 9, wherein the diagnostic agent is adapted to change a physical characteristic based upon at least one characteristic of the blood.

11. The hemostatic product of claim 10, wherein the physical characteristic comprises at least one of color and temperature.

12. The hemostatic product of claim 9, wherein the dextran support comprises electrospun dextran and wherein the hemostatic agent comprises at least one of fibrinogen and thrombin.

13. The hemostatic product of claim 9, wherein the fibrinogen is placed on the dextran support at a concentration of between about 20 and 60 milligrams per square centimeter of the dextran support, wherein the thrombin is placed on the dextran support at a concentration of between about 2 and 200 NIH Units per square centimeter of the dextran support and wherein the thrombin and fibrinogen are placed on the dextran support in a substantially uniform manner.
